(19) United States
(12) Patent Application Publication
(10) Pub. No.: US 2008/0188706 A1
(43) Pub. Date: Aug. 7, 2008

(54) Device for local treatment of human body parts

(76) Inventor: Cindy Gonet, South Dartmouth, MA (US)

Correspondence Address: HEDMAN & COSTIGAN P.C., 1185 AVENUE OF THE AMERICAS, NEW YORK, NY, 10036, US

(21) Appl. No.: 11/703,396

(22) Filed: Feb. 7, 2007

(51) Int. Cl. A61F 7/00; A61H 1/00; A61M 35/00; A61N 2/00

(52) U.S. Cl. 600/15; 128/100.1; 128/101.1; 601/46; 607/112

(57) ABSTRACT

The device according to the present invention is a device for the local treatment of human body parts which consists preferably of a pad of quasi-rectangular shape provided with at least one pouch adapted to contain means for treating the body part of interest and with a set of elastic straps and belts, adapted to position and secure said pad on the body part to be treated.

### FIELD OF THE INVENTION

The present invention refers to a device for the local treatment of human body parts.

The device according to the present invention is provided with one or more pouches for the insertion of treatment elements generating cold or heat, vibration, magnetic or electomagnetic fields and can be easily adjusted to be positioned on different parts of the human body thanks to a set of belts adapted to place and secure said device where it is needed.

### PRIOR ART

It's well known the local treatment of human body parts against discomfort and pain caused by stress, shocks, extreme environmental conditions and even neuro-vascular or skeletal/muscular abnormalities.

Remedies known in the art comprise devices, often shaped in bands and the like, adapted to be placed and secured on the body parts in need of treatment.

Said remedies range from rudimentary means such as towels or bags filled with ice etc. (difficult to be placed in an efficient and stable way on the body part to be treated) to bands particularly designed and shaped to be placed on selected parts of the body.

U.S. Pat. No. 5,400,617 discloses a device for the local cooling of human body parts shaped to be adjusted on athletes' neck and limbs which are particularly stressed or shocked.

U.S. Pat. No. 6,554,787 discloses a headband for treatment of headaches shaped to be worn around the head above the ears, covering the forehead, the sides and the back of the head.

None of the above devices is adapted to be placed in an easy and efficient way on different parts of the human body in order to be used for the treatment of several, different pain and stress conditions affecting a large number of human body parts such as head, forehead, face, neck, limbs, hands, wrists, elbows, feet, ankles, shoulders, chest and so on.

### SUMMARY OF THE INVENTION

An object of the present invention is a device for the local treatment of human body parts which consists preferably of a pad of quasi-rectangular shape provided with at least one pouch adapted to contain means for treating the body part of interest and with a set of elastic straps and belts, adapted to position and secure said pad on the body part to be treated.

### LIST OF THE FIGURES

FIG. 1 shows a preferred embodiment of the inner and outer layers of the first quasi-rectangular shaped pad.

FIG. 2a shows a preferred embodiment of the belts, straps and scarves adapted to be associated to the first quasi-rectangular shaped pad.

FIG. 2b shows a preferred embodiment of the second quasi-rectangular shaped pad.

FIG. 3 shows various examples of application of the device according to the present invention.

FIG. 4 shows a further example of application of the device according to the present invention.

FIG. 5 shows a further preferred embodiment of the device according to the present invention.

### DETAILED DESCRIPTION OF THE INVENTION

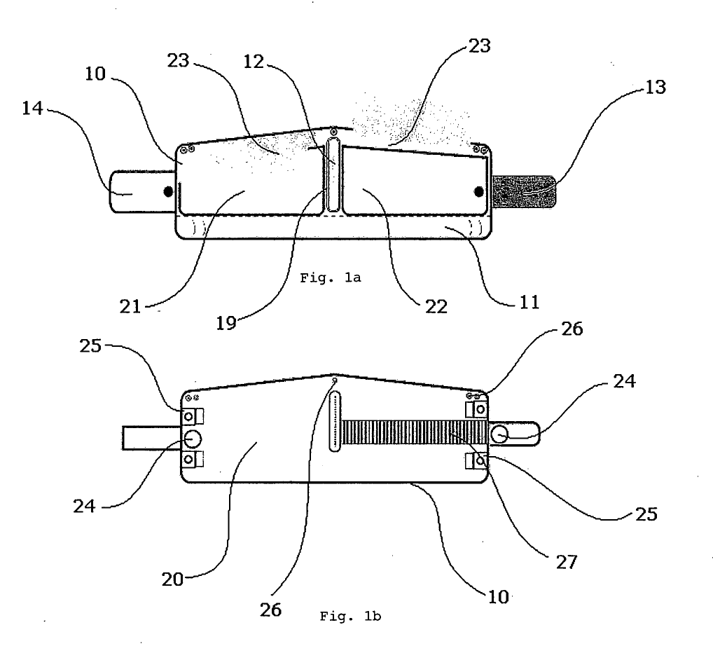

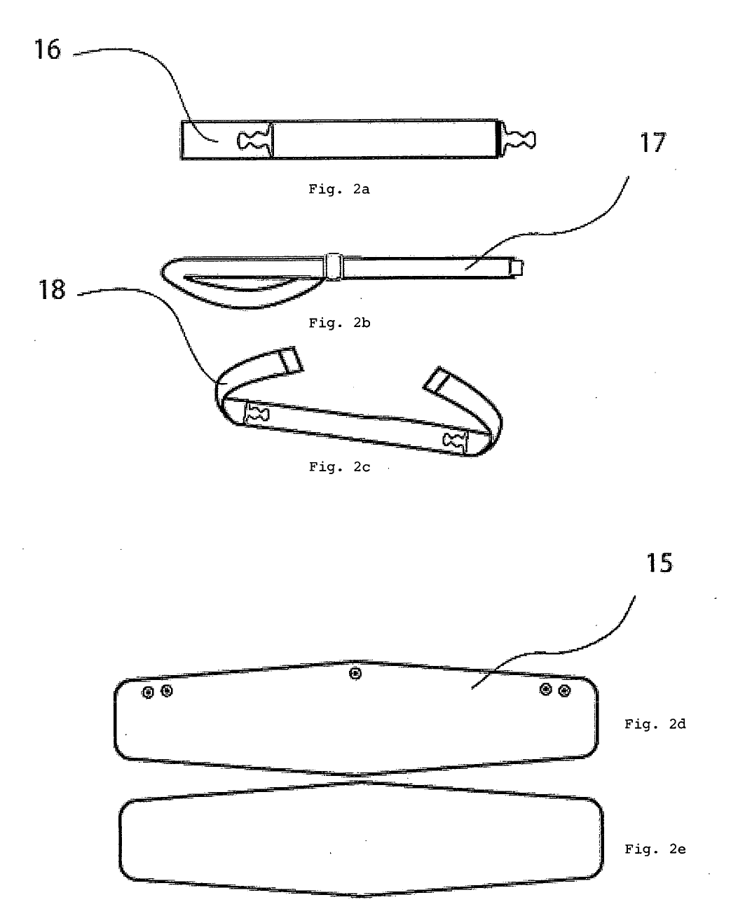

With reference to the enclosed FIGS. 1, 2a and 2b, a preferred embodiment of the present invention comprises:
a first quasi-rectangular shaped pad 10 provided with: at least an elastic strip 11 placed along one of its longest sides; at least one opening 12 shaped like a button hole and at least a tab 13, 14, preferably provided with means for attaching belts and analogous means for adjusting, securing and fastening and placed on each one of the pad's 10 shortest sides;
a set of belts 16, straps 17, scarves 18 and analogous means for adjusting, securing and fastening, adapted to be associated to said first quasi-rectangular shaped pad 10.
an optional second quasi-rectangular shaped pad 15 provided with means to be attached to said first quasi-rectangular shaped pad 10, said means being preferably snaps.

Said quasi-rectangular shaped pad 10 further comprises an inner layer 19 and an outer layer 20. Said inner layer 19 is provided with at least one pouch 21, 22 provided, in turn, with a preferably closeable opening 23 and adapted to contain means for the treatment of the body part of interest such as heating, cooling, vibratory, magnetic, electromagnetic etc. Said closeable openings are provided with reversible means of closing such as zippers, Velcro™ pads or any other suitable means. Furthermore, said inner layer preferably comprises at least one silicone strip or drop in order to avoid shifting of the pad on the human body.

Said outer layer 20 is provided with means for attaching belts, preferably comprising rivets 24, press-and-release connectors 25, snaps 26 and Velcro™ strips 27.

Said belts 16, straps 17 and scarves 18 are adapted to engage with said means for attaching belts 24, 25, 26, 27, with said opening 12 shaped like a button hole and with said tab 13, 14 and are provided with means which are adapted, in turn, to engage with each other in order to easily tighten said pad on or around the part to be treated and subsequently release it. These means can be chosen in the group comprising hooks, snaps, buckles, rivets, D-rings, Velcro™ pads, press-and-release connectors etc.

The preferably quasi-rectangular pad 10 can be suitably adjusted to be placed and secured on several body parts according to the treatment to be performed. Thanks to said means for attaching belts 24, 25, 26, 27, the user can advantageously place said quasi-rectangular pad 10 on the chosen body part in need of treatment exerting the desired compression. Moreover, by engaging said belts with said opening 12 shaped like a button hole, the user can advantageously reduce the length of said quasi-rectangular pad 10 thus achieving extra flexibility in placing it on the body part to be treated.

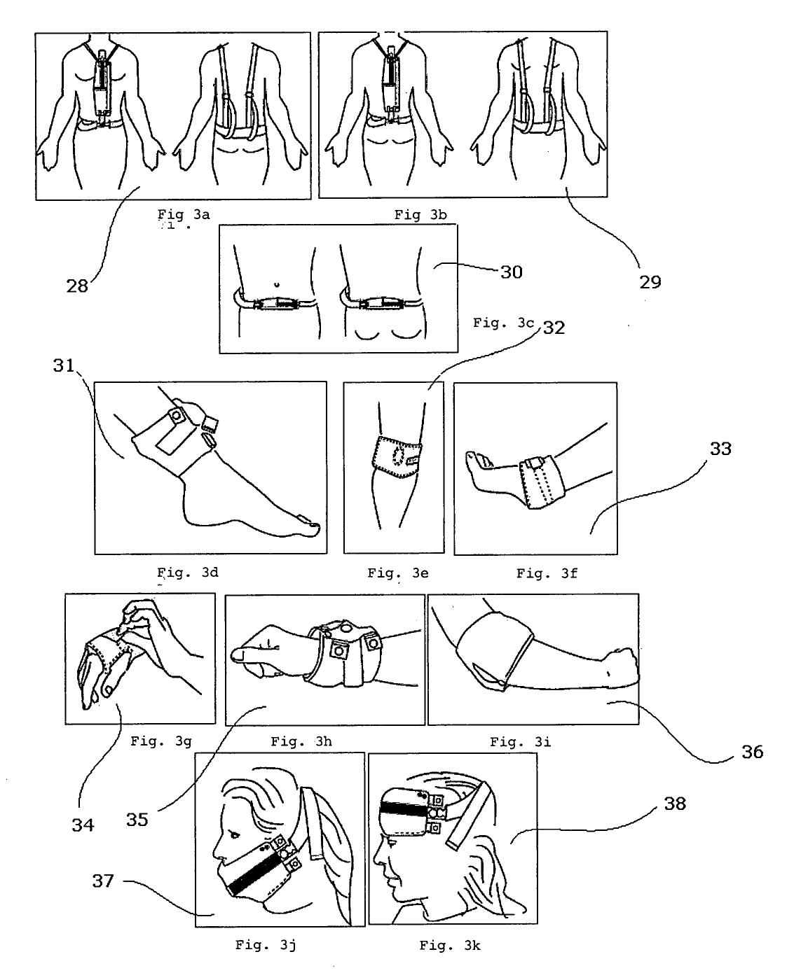

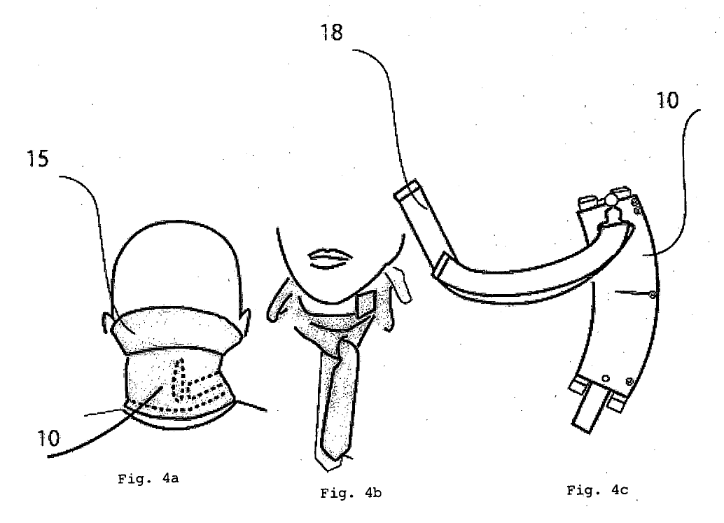

With reference to FIG. 3 said pad can be placed on the patient's chest 28, back 29 waist and abdomen 30, on his legs 31, knees 32 and ankles 33, on his hands 34 and wrists 35, on his elbows 36, on his mouth and chin 37, on his head and forehead 38 by suitable connection of said belts 16, straps 17 and scarves 18 with said opening 12 and with said tab 20. Moreover, with reference to FIG. 4, by connecting said first and said second quasi-rectangular pads 10, 15 together, the device according to the present invention can be adapted as a collar, wrapped around the user's neck using the scarf 18 enclosed in said set of belts and straps or it can be extended in a way to cover the chin and/or the nape.

The closable pouches 21, 22 of said pad 10 can be filled with means for heating, cooling, vibratory, magnetic, electromagnetic etc. or any combination of them. Heating and cooling means may comprise suitable chemicals, well known in the art, sealed in appropriate containers and the like, adapted to be refrigerated or heated.

Vibratory means may comprise a preferably rechargeable battery operated vibratory motor providing means of selecting and programming its oscillating motion.

Magnetic means may comprise permanent magnets, well known in the art, with different intensity of the generated magnetic field.

Electro-magnetic means may include electromagnetic field generators where the power and the frequency of the generated electromagnetic fields can be chosen and programmed.

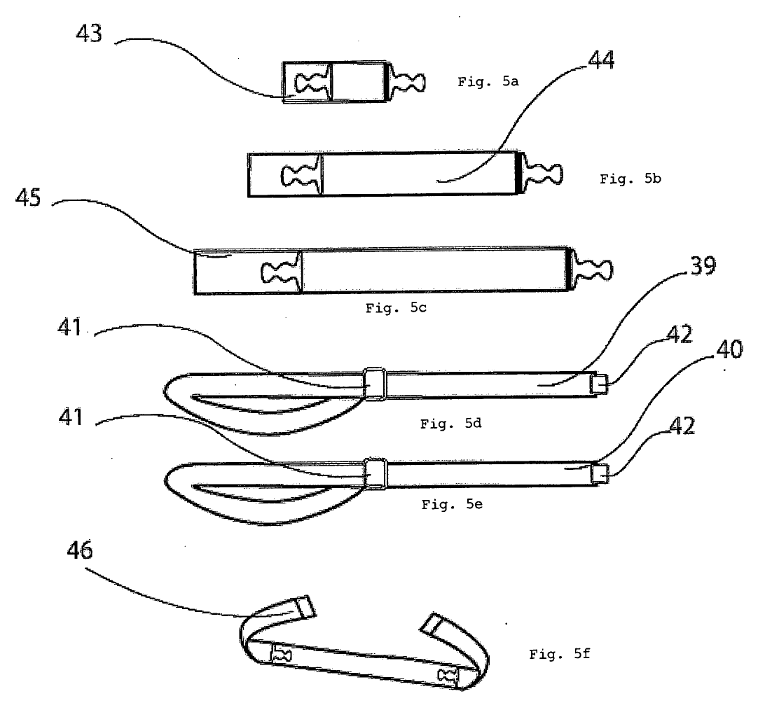

In a preferred embodiment of the present invention, displayed in FIG. 5, said set of belts 16, straps 17 and scarves 18 adapted to be associated to said first and second quasi-rectangular shaped pads 10, 15 comprises:
A pair of elastic straps 39, 40 provided with means to adjust their length 41 and with connectors 42 preferably of the press-and-release type.
Three belts, of different lengths, 43, 44, 45, preferably made of elastic Velcro™, each provided with at least two hooks, one fixed at one end, the other slidable along the belt length.
One scarf 46 provided with two slidable hooks

1. A device for the local treatment of human body parts comprising:
a first quasi-rectangular shaped pad 10 provided with means for attaching belts and analogous means for adjusting, securing and fastening;
a set of means for adjusting, securing and fastening, adapted to be associated to said first quasi-rectangular shaped pad 10.

2. The device according to claim 1 wherein said quasi-rectangular shaped pad 10 comprises at least an elastic strip 11 placed along one of its longest sides; at least one opening 12 shaped like a button hole and at least a tab 13, 14

3. The device according to claim 2 wherein said tab 13, 14, comprise means for attaching belts and analogous means for adjusting, securing and fastening and are placed on each one of the pad's 10 shortest sides.

4. The device according to claim 3 wherein said quasi-rectangular shaped pad 10 further comprises an inner layer 19 and an outer layer 20, said inner layer 19 being provided with at least one pouch 21, 22 adapted to contain means for the treatment of the body part of interest and with at least one silicone strip or drop in order to avoid shifting of the pad on the human body.

5. The device according to claim 4 wherein said means for the treatment of the body part of interest comprise means for heating and cooling, vibratory means, magnetic and electro-magnetic field generating means.

6. The device according to claim 5 wherein said heating and cooling means comprise suitable chemicals, sealed in appropriate containers and the like, adapted to be refrigerated or heated.

7. The device according to claim 5 wherein said vibratory means comprises a battery operated vibratory motor provided with means of selecting and programming its oscillating motion.

8. The device according to claim 5 wherein said magnetic means comprise permanent magnets.

9. The device according to claim 5 wherein said electro-magnetic means include electro-magnetic field generators where the power and the frequency of the generated electromagnetic fields can be chosen and programmed.

10. The device according to claim 5 wherein said at least one pouch 21, 22, comprises a closeable opening 23 provided with reversible means of closing such as zippers, Velcro™ pads or any other suitable means.

11. The device according to claim 6 wherein said outer layer 20 is provided with means for attaching said means for adjusting, securing and fastening.

12. The device according to claim 7 wherein said means for attaching said means for adjusting, securing and fastening, comprise rivets 24, press-and-release connectors 25, snaps 26 and Velcro™ strips 27.

13. The device according to claim 8 wherein said means for adjusting, securing and fastening comprise belts 16, straps 17 and scarves 18

14. The device according to claim 9 wherein said belts 16, straps 17 and scarves 18 are adapted to engage with said means for attaching belts 24, 25, 26, 27 with said opening 12 shaped like a button hole and with said tab 13, 14 and are provided with means which are adapted to engage with each other in order to easily tighten said pad on or around the part to be treated and subsequently release it.

15. The device according to claim 10 wherein said means which are adapted to engage with each other in order to easily tighten said pad on or around the part to be treated and subsequently release it, can be chosen in the group comprising hooks, snaps, buckles, rivets, D-rings and Velcro™ pads.

16. The device according to claim 11 further comprising a second quasi-rectangular shaped pad 15 provided with means to be attached to said first quasi-rectangular shaped pad 10.

17. The device according to claim 12 wherein said means to be attached to said first quasi-rectangular shaped pad 10 comprise snaps.

18. A device for the local treatment of human body parts comprising:
a first quasi-rectangular shaped pad 10 comprising: means for attaching belts and analogous means for adjusting, securing and fastening, said means for attaching belts being chosen in the group comprising rivets 24, press-and-release connectors 25, snaps 26 and Velcro™ strips 27; an inner layer 19 being provided with at least one silicone strip or drop in order to avoid shifting of the pad on the human body and with at least one pouch 21, 22 adapted to contain means for the treatment of the body part of interest, said at least one pouch being provided with a closeable opening 23 provided with reversible means of closing such as zippers, Velcro™ pads or any other suitable means; an outer layer 20 provided with rivets 24, press-and-release connectors 25, snaps 26 and Velcro™ strips 27 for attaching said means for adjusting, securing and fastening.
a set of means for adjusting, securing and fastening, adapted to be associated to said first quasi-rectangular shaped pad 10 comprising: a pair of elastic straps 39, 40 provided with means to adjust their length 41 and with connectors 42 of the press-and-release type; three belts, of different lengths 43, 44, 45, made of elastic Velcro™, each provided with at least two hooks, one fixed at one end, the other slidable along the belt length; one scarf provided with two slidable hooks.
a second quasi-rectangular shaped pad 15 provided with means to be attached to said first quasi-rectangular shaped pad 10, said means being preferably snaps.

19. The device according to claim 14 wherein said at least one pouch is adapted to contain means for the treatment of the body part of interest, said means comprising means for heating and cooling, vibratory means, magnetic and electro-magnetic field generating means.
